(19) United States
(12) Patent Application Publication
(10) Pub. No.: US 2007/0225738 A1
(43) Pub. Date: Sep. 27, 2007

(54) Aneurysm coil and method of assembly

(75) Inventor: Dharmendra Pal, Wilmington, VA (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/726,495

(22) Filed: Mar. 22, 2007

Related U.S. Application Data
(60) Provisional application No. 60/785,481, filed on Mar. 24, 2006.

(51) Int. Cl. A61B 17/08; A61M 29/00

(52) U.S. Cl. 606/151; 606/200

(57) ABSTRACT

An occlusion device for occluding blood flow in a vessel is provided, having a generally helical member with a pair of end portions and an intermediate portion therebetween. The intermediate portion defines a varying coil pitch so that the helical member has a varying stiffness along the intermediate portion.

### CROSS-REFERENCE TO RELATED APPLICATION

This patent application claims the benefit under 35 U.S.C. § 119(e) of U.S. Provisional Patent Application Ser. No. 60/785,481, filed Mar. 24, 2006 and entitled ANEURYSM COIL AND METHOD OF ASSEMBLY, the entire contents of which is incorporated herein by reference.

### BACKGROUND

The invention relates generally to medical devices. More specifically, the invention relates to occlusion devices for occluding a dilatation area of a body vessel forming an aneurysm and methods for assembling occlusion devices.

Aneurysms, e.g., cerebral aneurysms, typically are formed as a result of the dilatation of a weakened wall of a body vessel, such as an artery or a vein, or the heart. Chief signs of an arterial aneurysm are the formation of a pulsating tumor, and often a bruit (aneurismal bruit) heard over a swelling. Often aneurysms take on a dome shape to define a sac at the weakened or dilatation area of the body vessel. The dome-shaped aneurysm includes a neck portion extending from the body vessel and an opening defined by the neck that permits blood flow through the neck and into the sac.

Untreated aneurysms may burst or rupture, thereby causing severe pain and bleeding and potentially causing death. Additionally, untreated aneurysms may cause other complication, such as the formation of blood clots at the aneurysm. Blood clots may break off the aneurysm, travel through the blood stream, and cause severe damage such as a stroke.

Currently, there are a number of existing methods for the treatment of aneurysms. For example, one method involves an open surgical procedure in which, under microscopic dissection, a small vascular clip is placed across the neck of the aneurysm thereby excluding it from the circulation through the body vessel. However, treatment with surgery involves its inherent risks. Thus, many practitioners and patients prefer to avoid treatment with surgery when possible.

In another method, treatment involves an endovascular or “closed” approach in which a microcatheter is navigated from the femoral artery in the groin area into the cerebral vessels, allowing the placement of a helical wire into the dome of the aneurysm. Under x-ray guidance, the helical wire is packed into the aneurysm, filling up its volume and thereby preventing blood from entering. More specifically, the helical wire is advanced into the dome of the aneurysm until the distal tip of the wire contacts the wall of the dome, thereby creating a compression tension in the helical wire and causing the helical wire to buckle and fold. As a greater length of the wire is fed into the dome of the aneurysm, more buckles are formed and the wire becomes more intertwined and more tightly-compacted.

However, it may be difficult or time-consuming to pack the helical wire within the dome of the aneurysm in a compact manner so as to effectively prevent blood from entering the dome. For example, the helical wire may not buckle in a desired location along the length thereof, thereby permitting the formation of unoccluded pockets within the aneurysm dome or the formation of a generally loosely-packed helical wire. The unoccluded pockets and/or loosely-packed wire may lead to an undesirably high blood flow into the aneurysm dome.

Thus, there is a need to improve the current devices and methods for treating aneurysms, for example cerebral aneurysms, by more-effectively reducing blood flow into the aneurysm.

### BRIEF SUMMARY OF THE INVENTION

One embodiment of the present invention is an occlusion device for occluding blood flow in a vessel, having a generally helical member with a pair of end portions and an intermediate portion therebetween. The intermediate portion defines a varying coil pitch so that the helical member has a varying stiffness along the intermediate portion. For example, the intermediate portion defines a first portion having a first coil pitch and a second portion having a second coil pitch that is larger than the first coil pitch so that the second portion has a higher flexibility than the first portion. In other words, the force required to bend or buckle the second portion is less than the force required to bend or buckle the second portion. Therefore, during delivery into the vessel, the occlusion device has a greater tendency to bend or buckle at a point along the second portion than at a point along the first portion.

In one aspect of the invention, the helical member is a wire having a generally rectangular cross-section. For example, the rectangular cross-section has a width between 0.002 and 0.004 inches and a height between 0.0005 and 0.002 inches. Additionally, the helical member preferably has an outer diameter between 0.005 and 0.05 inches.

In another aspect of the invention, the helical member is at least partially covered by a connective tissue coating, such as an extracellular matrix. The extracellular matrix is preferably small intestinal submucosa.

Another embodiment of the present invention is a system for occluding blood flow in a vessel, including a catheter having a passageway that extends from its proximal end to its distal end and a generally helical member configured to be selectively disposed within the passageway and to be deployed from the distal end of the catheter. The helical member includes a first end portion, a second end portion, and an intermediate portion therebetween having a varying stiffness along a length thereof.

Yet another embodiment of the present invention is a method of assembly of an occlusion device, including the steps of providing a wire extending generally along an axial length and introducing a residual stress to the wire along at least a portion of the axial length so as to form a helical portion of the wire.

In one aspect of the invention, the residual stress introduced to the wire varies along the axial length so that the helical portion of the wire defines a varying coil pitch.

In another aspect, the step of introducing the residual stress includes engaging the wire with a coiling tool and translating at least one of the wire and the coiling tool so that the wire and the coiling tool move with respect to each other. In yet another aspect, the pitch of the coiled portion is varied by varying the angle of the coiling tool with respect to the wire or by varying the magnitude of the engagement force between the coiling tool and the wire.

Further objects, features, and advantages of the present invention will become apparent from consideration of the following description and the appended claims when taken in connection with the accompanying drawings.

### BRIEF DESCRIPTION OF THE DRAWINGS

FIG. 1 is a side environmental view of a body vessel, such as a cerebral vessel, having an unoccluded dome-shaped aneurysm;

FIG. 2 is a side environmental view of a catheter and an occlusion device in accordance with one embodiment of the present invention, where the occlusion device is in the process of being deployed within the aneurysm by the catheter;

FIG. 2A is an enlarged view taken around line 2A in FIG. 2 showing a folded portion of the occlusion device;

FIG. 3 is a side environmental view of the catheter and the occlusion device in FIG. 2, where the occlusion device is substantially completely deployed within the aneurysm;

FIG. 4 is an enlarged cross-sectional view of the occlusion device shown in FIG. 2;

FIG. 4A is an enlarged view taken around line 4A in FIG. 4 showing a varying coil pitch of the occlusion device;

FIG. 5 is a cross-sectional view of an occlusion device in accordance with another embodiment of the present invention; and

FIG. 6 is a side view of a coiling tool used in a method of assembly of an occlusion device in accordance with one embodiment of the present invention.

### DETAILED DESCRIPTION OF THE INVENTION

Embodiments of the present invention generally provide an occlusion device, an occlusion system, and a method of assembly of an occlusion device for occluding a dilatation area of a body vessel formed by an aneurysm. The embodiments solve the concerns of current aneurysm treatments, such as the formation of unoccluded pockets and/or a loosely-packed helical wire within the aneurysm. Rather, embodiments of the present invention provide a tightly-packed helical wire to substantially completely occlude the aneurysm.

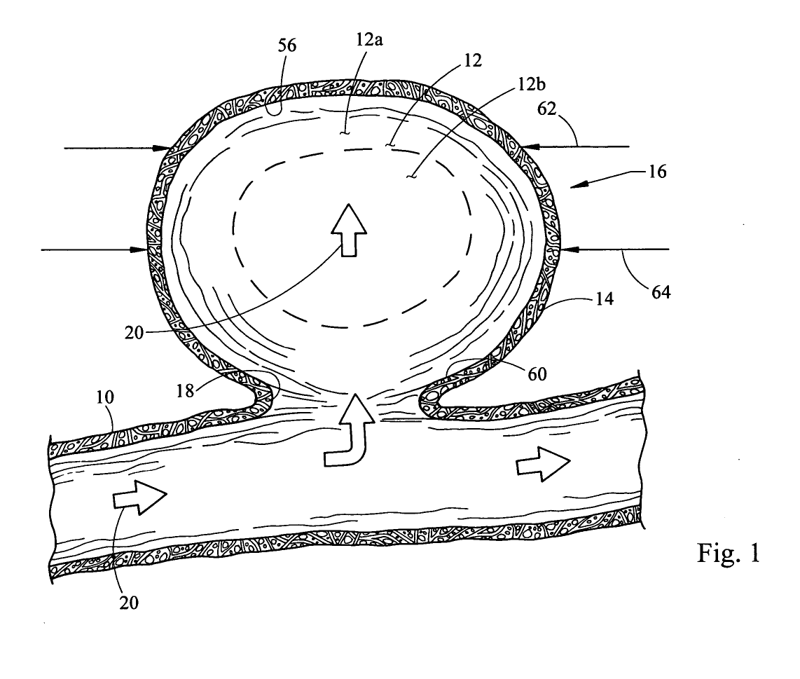

FIG. 1 illustrates a body vessel 10 having a dilatation area 12 formed by an aneurysm 14. As shown, the aneurysm 14 is formed of a sac or dome-like structure 16 having a neck 18 extending from the body vessel 10 to define the dilatation area 12. As is known, an aneurysm is formed by a weakened or dilatation area on a body vessel. The dilatation area may be congenital or caused by high blood pressure. Pressure from blood flow 20 causes the dilatation area 12 to dilate and expand to form the abnormal sac or dome-like structure 16.

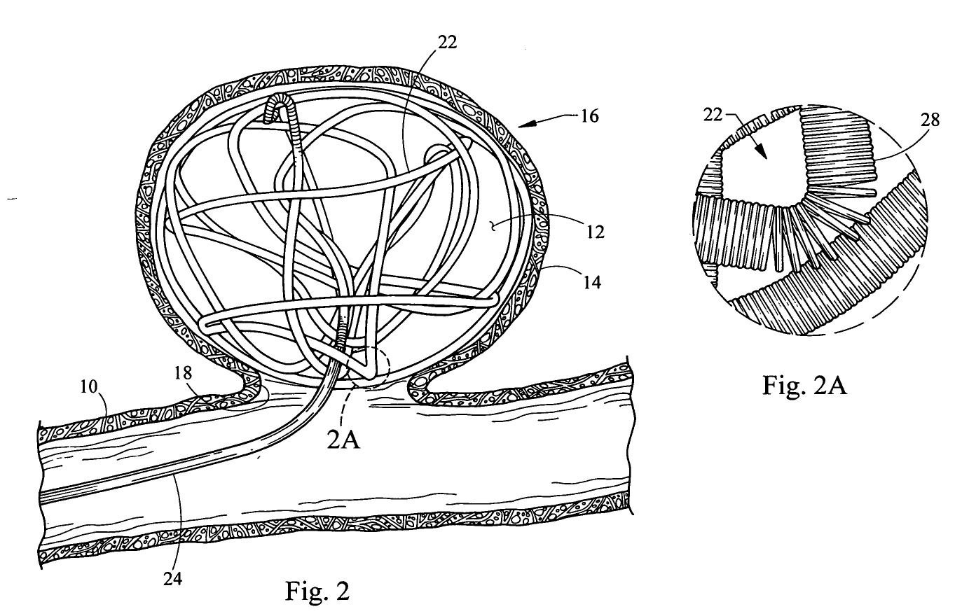

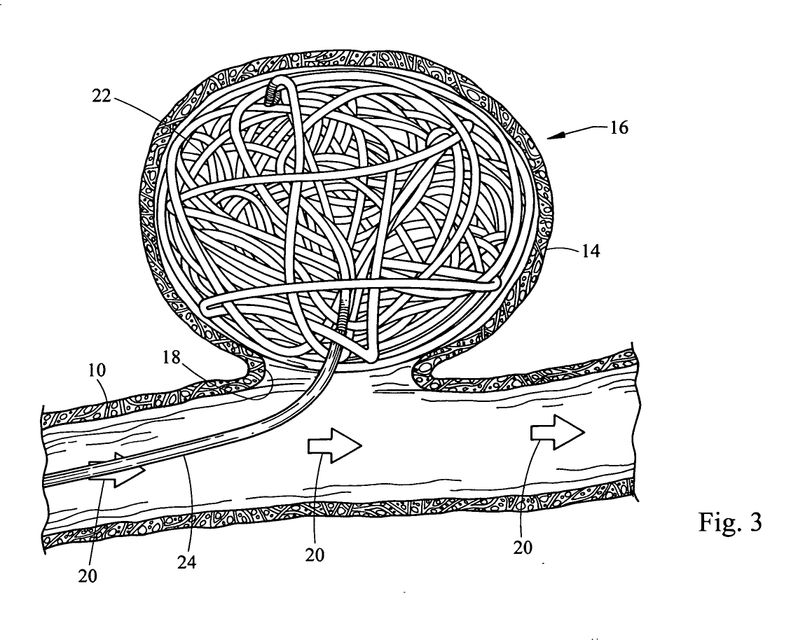

FIGS. 2 and 2A illustrate an occlusion device 22 that is partially-deployed within the dilatation area 12 of the body vessel 10 so that the aneurysm 14 may be occluded and the blood flow 20 may be prevented from entering the dilation area 12. The occlusion device 22 is deployed into the dilation area 12 by a catheter 24 that is preferably inserted percutaneously into the body vessel 10 and is then positioned near the neck 18 or within the dilation area 12 of the aneurysm 14. Once the catheter 24 is positioned as desired, the occlusion device 22 is advanced into the dilation area 12 and becomes folded and intertwined to generally block blood flow into the aneurysm 14. As the occlusion device 22 is further advanced from the catheter, the dilation area 12 is further occluded and the blood flow in the aneurysm 14 is further reduced. Once the occlusion device 22 is completely deployed within the aneurysm 14, the dilation area 12 is substantially completely filled with the occlusion device 22 as shown in FIG. 3.

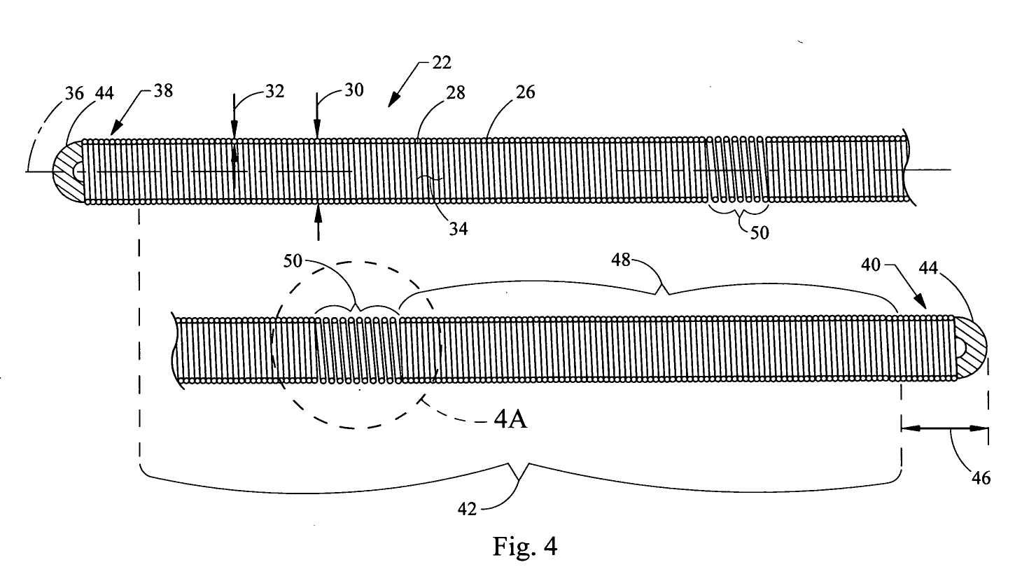

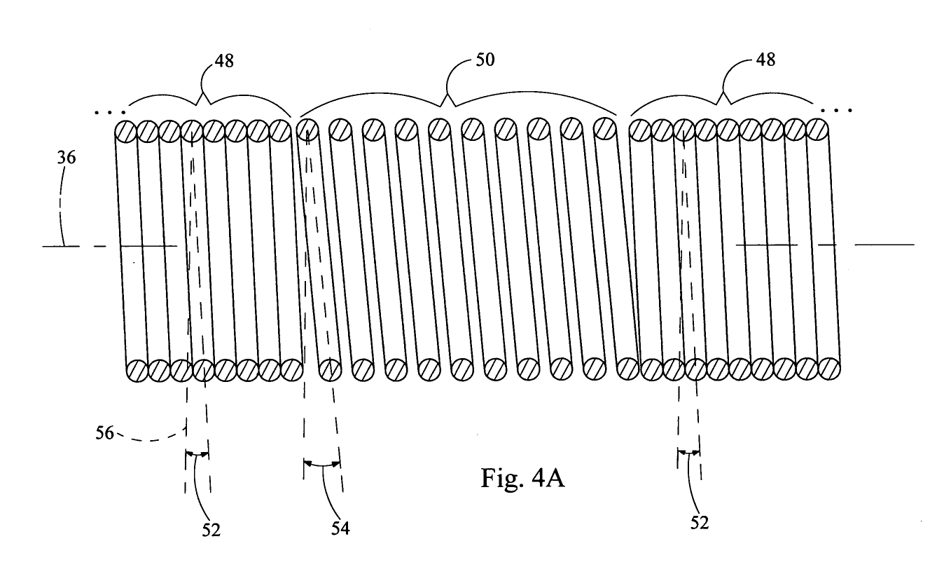

As shown in FIG. 4, the occlusion device 22 is a generally helical member 26 formed by a generally tightly-coiled wire 28. More specifically, the wire 28 is coiled so that the helical member 26 defines an outer diameter 30 that is significantly larger than a cross-sectional thickness 32 of the wire 28 and defines a passageway 34 extending along a longitudinal axis 36 of the occlusion device 22. For example, in one preferred embodiment the helical member 26 outer diameter 30 is between 0.005 and 0.05 inches and the wire 28 cross-sectional thickness 32 is between 0.0005 and 0.005 inches. The wire 28 is made of any suitable material, such as nitinol or stainless steel.

The generally tightly-coiled nature of the helical member 26 provides axial strength for the occlusion device 22 so that it can be advanced through the catheter 24. More specifically, when a force is applied to the occlusion device 22 along the longitudinal axis 36, the force is transferred between adjacent coils of the wire and the occlusion device 22 is advanced. However, the coiled nature of the helical member 26 also provides a suitable flexibility so that the occlusion device 22 is able to be folded and intertwined as shown in FIG. 2A.

The helical member 26 includes a pair of end portions 38, 40 and an intermediate portion 42 extending therebetween. More specifically, the first end portion 38 is the proximal end portion of the occlusion device 22 and the second end portion 40 is the distal end portion of the occlusion device 22 that is first advanced into the aneurysm 14. Each of the end portions 38, 40 preferably includes a cap portion 44 that is coupled to the helical member 26 to reduce the likelihood of puncturing the wall of the aneurysm 14. As used herein, the term “intermediate portion” 42 is generally defined as the portion of the helical member 26 that is likely to be subject to bending or folding forces while the occlusion device 22 is being deployed within the aneurysm 14. The end portions 38, 40 have a relatively short length 46, so when the occlusion device 22 is advanced forward the end portions 38, 40 typically slip along the walls of the aneurysm 14 rather than bend or fold.

The intermediate portion 42 of the helical member 26 includes a plurality of first zones 48 having a relatively high stiffness to improve the pushability of the occlusion device 22 and a plurality of second zones 50 having a lower stiffness than the first zones 48 to promote folding and intertwining of the occlusion device 22 within the aneurysm 14. The varying stiffness of the helical member 26 in the figures is due to a varying coil pitch along the longitudinal axis 36 of the occlusion device 22. For example, as shown in FIG. 4A, the coils in the first zones 48 have a first coil pitch 52 between 0.001 and 5 degrees and the coils in the second zones 50 have a second coil pitch 54 between 5 and 10 degrees. The exemplary coil pitches referenced herein are measured with respect to the radial direction 56, which is perpendicular to the longitudinal axis 36.

Due to the varying stiffness along the length thereof, the helical member 26 will have a tendency to bend at a point along one of the second zones 50 rather than at a point along one of the first zones 48 when the occlusion device 22 is advanced into the aneurysm 14. For example, when the helical member 26 is being advanced into the dilation area 12 it will contact the inner wall thereof and cause compression forces along the longitudinal axis 36 of the occlusion device 22, thereby causing a buckling and/or bending at a point along the helical member 26. More specifically, the buckling and/or bending will be more likely to occur at a point along one of the second zones 50 than at a point along one of the first zones 48. Therefore, when designing the occlusion device 22, the design characteristics of the first and second zones 48, 50 can be configured to promote a particularly desirable bending pattern within the aneurysm 14.

As a first example, the distal portion of the helical member 26 has a relatively small number of second zones 50 so that the occlusion device 22 tends to fill the outer areas (generally designated by 12A in FIG. 1) of the dilation area 12 before filling the inner areas (generally designated by 12B in FIG. 1). More specifically, if the distal portion of the helical member 26 includes a small number of second zones 50, then the distal portion of the helical member 26 will tend to lie along the inner surface of the aneurysm 14 and encircle the outer areas 12A of the dilation area 12 rather than folding and extending through the inner areas 12B. Therefore, the first exemplary occlusion device 22 will tend to occlude the outer areas 12A before the inner areas 12B and will reduce the number of unoccluded cavities in the outer areas 12A of the dilation area 12.

As a second example, the frequency of the second zones 50 increases from the distal portion to the proximal portion of the occlusion device 22 so that the helical member 26 progressively fills the outer areas 12A before the inner areas 12B in a manner similar to the first example.

As a third example, the frequency of the second zones 50 may be relatively constant along the length of the occlusion device 22 so that the dilation area 12 may be progressively filled the top wall 58 to the bottom wall 60 of the aneurysm 14 (as indicated in FIG. 1). More specifically, in this example, the catheter 24 is preferably inserted into the dilation area and positioned near the top wall 58. Then, as the occlusion device 22 is advanced, the catheter 24 is slowly withdrawn away from the top wall 58 as the area immediately above the catheter is filled with the helical member 26. In this example, the distance between each of the second zones 50 is preferably generally equal to the median width 62 or the maximum width 64 of the dilation area 12 (as indicated in FIG. 1).

In a fourth example, similar to the third example, the distance between each of the second zones 50 becomes progressively larger from the distal portion to the midpoint of the helical member 26 and then becomes progressively smaller from the midpoint of the helical member 26 to the proximal portion. More specifically, the distance between the second zones 50 along the distal portion of the helical member 26 is generally equal to the width of the dilation area 12 near the top wall 56 so that the distal portion of the helical member 26 folds near the walls of the aneurysm 14 and minimizes unoccluded pockets in the upper portion of the dilation area 12. Similarly, the distance between the second zones 50 at the midpoint of the helical member 26 is generally equal to the maximum width 64 of the dilation area 12 and the distance between the second zones 50 at the proximal portion of the helical member 26 is generally equal to the width of the dilation area 12 near the bottom wall 60.

In a fifth example, the second zones 50 are randomly spaced from each other along the length of the helical member 26 to create random folds within the aneurysm 14.

In a sixth example, the second zones 50 are spaced from each other based on the results of experimental trials.

In a seventh example, each or many of the second zones 50 have coil pitches that vary from each other and/or each have varying lengths.

In an eighth example, all or some of the second zones 50 have a connective tissue 66 disposed thereon to promote biological connections between the inner walls of the aneurysm and the helical member 26. This configuration promotes connection points at the folding points of the helical member 26 and reduces unoccluded spaces near the inner wall of the aneurysm 14. More specifically, the connective tissue induces tissue growth at the connection points, wherein host cells of the body vessel become stimulated to proliferate and differentiate into site-specific connective tissue structures.

Reconstituted or naturally-derived collagenous materials can be used as the connective tissue 66 in the present invention. Such materials that are at least bioresorbable will provide advantage in the present invention, with materials that are bioremodelable and promote cellular invasion and ingrowth providing particular advantage.

Suitable bioremodelable materials can be provided by collagenous extracellular matrix materials (ECMs) possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics or thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient.

Submucosa or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

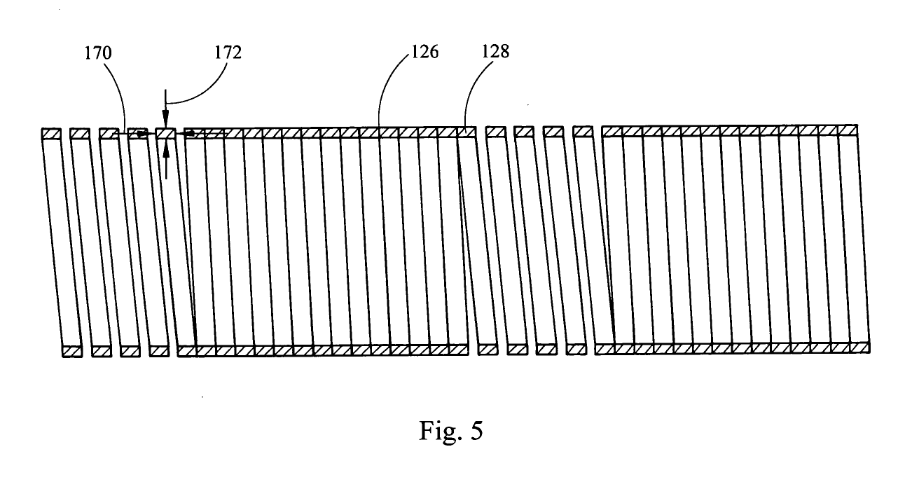

In a ninth example, as shown in FIG. 5, the helical member 126 is defined by a wire 128 having a generally rectangular cross-section. The wire 128 in FIG. 5 has a width 170 between 0.002 and 0.004 inches and a height between 0.0005 and 0.002 inches.

In a tenth example, the second zones 50 are formed while the occlusion device 22 is inside of the body vessel 10. For example, the second zones 50 may be formed by the tip of the catheter 24 under the direction of the medical professional. More specifically, the distal end of the catheter 24 may include a deforming component that is able to locally stretch or otherwise deform the coiled the portion when desired. The deforming component is able to be controlled by the medical professional during the medical procedure so that the second zones 50 are formed as desired. For example, the medical professional is able to observe the action of the occlusion device 22 via an x-ray machine and to selectively deform portions of the occlusion device 22 in order to tightly pack the aneurysm 14. The deforming component may additionally, or alternatively, sever the coiled portion 26 once the aneurysm has been sufficiently occluded with a portion of the length of the helical member 26. As one example, the deforming component may include a pinching or squeezing assembly that flattens a portion of the coiled member. Alternatively, the deforming component may include components for stretching a localized portion of the coiled member. As yet another alternative, an electrical current may be used to deform or reshape localized portions of the coiled member.

The above examples may be combined with each other to create additional design configurations. Additionally, the above examples are to be considered merely for exemplary purposes rather than as limitations on the present invention.

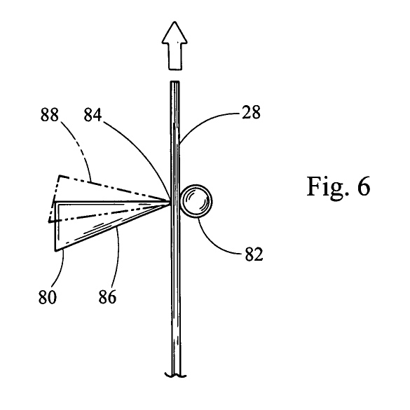

A method of assembly of an occlusion device in accordance with an embodiment of the present invention will now be discussed. The occlusion device 22 is preferably formed by introducing a residual stress to the wire 28 along at least a portion of the axial length so as to form the helical member 26, 126. For example, as shown in FIG. 6, this step may be accomplished by engaging the wire 28 with a coiling tool, so that an engagement force occurs therebetween, and translating the wire 28 with respect to the coiling tool 80, so that the coiling tool 80 creates slight surface deformations and causes a curling action of the wire 28. The coiling tool 80 is preferably a high-strength metal component with a generally tapered coiling edge 84. To maintain the engagement force between the respective components 28, 80, a roller 82 is positioned on the opposite side of the wire 28 from the coiling tool 80. This action is not unlike the effect of dragging the edge of a scissors blade across a piece of gift wrapping ribbon to form decorative curls therein.

The coil pitch can be varied by varying the engagement force between the respective components 28, 80 or by varying the angle of the coiling tool 80 with respect to the wire 28. For example, the coiling tool 80 in FIG. 6 is shown in a first position 86 by solid lines and is shown in a second position 88 by phantom lines. The magnitude of the engagement force and/or the coiling tool 80 angle may be controlled by any appropriate means, such as an electronically-based control system or by hand-controlled components. As another embodiment, the coil pitch can be varied by varying the translational speed of the wire 28 with respect to the coiling tool 80.

While the present invention has been described in terms of preferred embodiments, it will be understood, of course, that the invention is not limited thereto since modifications may be made to those skilled in the art, particularly in light of the foregoing teachings.

1. An occlusion device for occluding blood flow in a vessel, comprising a generally helical member having a pair of end portions and an intermediate portion therebetween, the intermediate portion defining a varying coil pitch so that the helical member has a varying stiffness along the intermediate portion.

2. An occlusion device as in claim 1, wherein the helical member includes a plurality of first zones each having a first stiffness and a plurality of second zones each having a second stiffness greater than the first stiffness so the helical member is more flexible in the second zones than in the first zones.

3. An occlusion device as in claim 2, the plurality of first zones each having a coil pitch less than or equal to 5 degrees and the plurality of second zones each having a coil pitch greater than or equal to 5 degrees.

4. An occlusion device as in claim 2, wherein the vessel includes a portion to be occluded by the occlusion device, and wherein a pair of adjacent second zones of the helical member are spaced apart from each other by a distance generally equal to a diameter of the portion of the vessel to be occluded by the occlusion device.

5. An occlusion device as in claim 4, wherein one of the end portions and one of the plurality of second zones are spaced apart from each other by a distance generally equal to the diameter of the portion of the vessel to be occluded by the occlusion device.

6. An occlusion device as in claim 1, wherein the helical member is defined by a wire having a generally rectangular cross-section.

7. An occlusion device as in claim 6, wherein the rectangular cross-section has a width between 0.002 and 0.004 inches and a height between 0.0005 and 0.002 inches.

8. An occlusion device as in claim 1 wherein the helical member is at least partially covered by a connective tissue coating.

9. An occlusion device as in claim 1 wherein the helical member is made of nitinol.

10. A system for occluding blood flow in a vessel comprising:
a catheter with a proximal end and a distal end, the catheter having a passageway that extends from the proximal end to the distal end; and
a generally helical member configured to be selectively disposed within the passageway and to be deployed from the distal end of the catheter, the helical member having a first end portion, a second end portion, and an intermediate portion therebetween, the intermediate portion having a varying stiffness along a length thereof.

11. A system as in claim 10, wherein the helical member includes a plurality of first zones each having a first stiffness and a plurality of second zones each having a second stiffness greater than the first stiffness so the helical member is more flexible in the second zones than in the first zones.

12. A system as in claim 11, the plurality of first zones each having a coil pitch less than or equal to 5 degrees and the plurality of second zones each having a coil pitch greater than or equal to 5 degrees.

13. A system as in claim 11, wherein the vessel includes a portion to be occluded by the occlusion device, and wherein a pair of adjacent second zones of the helical member are spaced apart from each other by a distance generally equal to a diameter of the portion of the vessel to be occluded by the occlusion device.

14. A system as in claim 13, wherein one of the end portions and one of the plurality of second zones are spaced apart from each other by a distance generally equal to the diameter of the portion of the vessel to be occluded by the occlusion device.

15. A system as in claim 10, wherein at least one of the plurality of second zones is at least partially covered by a connective tissue coating.

16. A method of assembly of an occlusion device comprising:
providing a wire extending generally along an axial length; and
introducing a residual stress to the wire along at least a portion of the axial length so as to coil the wire into a coiled portion of the occlusion device; and
varying a magnitude of the residual stress introduced to the wire along the axial length of the wire so that the coiled portion of the occlusion device defines a varying coil pitch along the axial length of the wire.

17. A method of assembly as in claim 16, where the step of introducing the residual stress includes:
engaging the wire with a coiling tool so that an engagement force occurs therebetween; and
translating at least one of the wire and the coiling tool so that the wire and the coiling tool move with respect to each other.

18. A method of assembly as in claim 17, wherein the step of engaging the wire with the coiling tool includes engaging the wire with a generally tapered coiling edge.

19. A method of assembly as in claim 18, wherein the step of engaging the wire with the coiling tool includes varying an angle of the coiling tool with respect to the wire so that the coiled portion of the occlusion device defines a varying coil pitch.

20. A method of assembly as in claim 18, wherein the step of engaging the wire with the coiling tool includes varying an engagement force between the coiling tool and the wire so that the coiled portion of the occlusion device defines a varying coil pitch.
